(19) United States
(12) Patent Application Publication
(10) Pub. No.: US 2022/0137612 A1
(43) Pub. Date: May 5, 2022

(54) TRANSFORMER FAULT DIAGNOSIS AND POSITIONING SYSTEM BASED ON DIGITAL TWIN

(71) Applicant: WUHAN UNIVERSITY, Hubei (CN)

(72) Inventors: Yigang HE, Hubei (CN); Jiajun DUAN, Hubei (CN); Xiaoxin WU, Hubei (CN); Liulu HE, Hubei (CN); Xiaoyan Liu, HUBEI (CN)

(73) Assignee: WUHAN UNIVERSITY, HUBEI (CN)

(21) Appl. No.: 17/509,065

(22) Filed: Oct. 25, 2021

(30) Foreign Application Priority Data
Nov. 5, 2020 (CN) 202011220442.9

(51) Int. Cl. G05B 23/02; G06N 7/00; G01R 31/62; G01N 33/28; G01J 5/00

(52) U.S. Cl. CPC G05B 23/0224; G06N 7/005; G01J 2005/0081; G01N 33/2841; G01J 5/00; G01R 31/62

(57) ABSTRACT

A transformer fault diagnosis and positioning system based on a digital twin is provided and includes the following. A communication sensing module, which is configured to transmit bottom-level monitoring data obtained from a device entity to a system support module. The system support module, which is configured to receive and preprocess bottom-level monitoring data, and store various data, models and expert systems. A dynamic twin module, which is configured to analyze a multi-dimensional probability status of a device fault, construct a dynamic degradation model of different health states, and realize model correction through human-computer interaction, real-time measurement, and dynamic update subsequently. A decision-making diagnosis module, which is configured to construct a digital twin of data to be diagnosed, to realize diagnosis and positioning of the health state. A user interface module to display a diagnosis result and related information through a UI interface, to provide further decision-making and human-computer interaction.

### CROSS-REFERENCE TO RELATED APPLICATION

This application claims the priority benefit of China application serial no. 202011220442.9, filed on Nov. 5, 2020. The entirety of the above-mentioned patent application is hereby incorporated by reference herein and made a part of this specification.

### BACKGROUND

### Technical Field

This disclosure belongs to the field of fault diagnosis of a power device, and in particular, relates to a transformer fault diagnosis and positioning system based on a digital twin.

### Description of Related Art

With the introduction of Internet of Things in electric power, the electrical power system is gradually presenting an open, flat, decentralized, and blurring of boundaries development. Accurately sensing and extracting device state information and extrapolating system trends are important prerequisites for realizing intelligent and optimal dispatching of the power system. However, current understanding of diagnosis of a physical device in the power system has the following issues. (1) There are barriers between historical documents and various types of monitoring information, and there is a lag in updating system information, while an operating state of the device itself is constantly evolving. (2) The device information may be inaccurate, such as a change in device environment and missing or abnormal measurement data. (3) Low intelligence level of operation and maintenance management, singular data source, and difficulty in realizing training of an intelligent diagnosis algorithm through a small amount of data.

Digital Twin (DT) technology uses modern information technology to fully tap/utilize big data resources, construct a digital virtual entity, and establish a “mirror” corresponding to a physical entity, thereby helping people to better regulate and make decisions in the process of power equipment health management. A transformer is a key device in the power system, and its fault diagnosis has always been the focus of research. Constructing a digital twin for panoramic monitoring and diagnosis of the transformer may integrate multiple types of information, make up for sample shortages, fuse diagnosis results, provide comprehensive decision-making, enhance safety of the power system, and play a demonstrative role in the diagnosis of other devices. Therefore, it is important and widely promising to explore the digital twin system of a power device represented by the transformer.

### SUMMARY

This disclosure proposes a transformer fault diagnosis and positioning system based on a digital twin, which is capable of improving integration level, intelligence, and diagnostic accuracy of the fault diagnosis method of a power device represented by a transformer.

The disclosure provides the transformer fault diagnosis and positioning system based on the digital twin, which includes the following.

A communication sensing module, which is configured to directly obtain bottom-level monitoring data from a device entity, and transmit the obtained bottom-level monitoring data to an upper-level system support module.

The system support module, which is configured to receive and preprocess the bottom-level monitoring data from the communication sensing module, and store various data, models and expert systems.

A dynamic twin module, which is configured to analyze a multi-dimensional probability status of a device fault, construct a degradation model based on different fault types and locations, and realize dynamic modeling of different health states of the transformer, and then further realizes model correction through human-computer interaction, real-time measurement, and dynamic update.

A decision-making diagnosis module, which is configured to integrate the information from the system support module and the dynamic twin module with data to be diagnosed, and use digital twin technology to construct a digital twin of the data to be diagnosed, and realize diagnosis positioning.

In some embodiments, the system further includes a user interface module, which is configured to display a diagnosis result and related information through a user interface, and provide further decision-making options and human-computer interaction.

In some embodiments, the communication sensing module includes a monitoring data acquisition module for a dissolved gas in transformer oil, a transformer temperature monitoring module, a transformer winding deformation monitoring module, and a transformer partial electrical discharge monitoring module.

In which, the monitoring data acquisition module for the dissolved gas in the transformer oil is configured to acquire concentration data of the dissolved gas in the transformer oil, the transformer temperature monitoring module is configured to monitor an internal hot spot temperature and external infrared thermography, the transformer winding deformation monitoring module is configured to monitor a transformer winding deformation status, and the transformer partial electrical discharge monitoring module is configured to monitor a partial electrical discharge status of the transformer.

In some embodiments, the system support module is configured to analyze the bottom-level monitoring data coming from the communication sensing module using a signal processing method, which includes data cleaning, feature extraction, labeling, and structuring.

In some embodiments, the system support module includes the following components. A transformer ledger knowledge map module, an expert system module for the dissolved gas in the transformer oil, a transformer temperature distribution diagnosis rule module, a transformer winding deformation simulation and diagnosis module, and a partial electrical discharge frequency spectrum characteristic analysis module.

The transformer ledger knowledge map module is configured to store transformer historical ledger information, the expert system module for the dissolved gas in the transformer oil is configured to store an expert system, the transformer temperature distribution diagnosis rule module is configured to store a temperature distribution diagnosis rule, the transformer winding deformation simulation and diagnosis module is configured to store a simulation model of the transformer, the partial electrical discharge frequency spectrum characteristic analysis module is configured to analyze the fault according to a partial electrical discharge frequency spectrum characteristic.

In some embodiments, the information stored by the transformer ledger knowledge map module includes a device entity map, which includes rated/operating voltage, power, frequency, capacity, structural parameters, materials, working location, geographical information, and environmental temperature and humidity of the transformer.

A device case map, which includes fault history case records and handling methods of a similar transformer, and calculates a similarity between the two devices through structural feature matching.

A business logic map, which includes a handling plan or a procedure of the transformer, containing general operating principles, fault causes, and handling points.

A concept map, which is intermediate information that connects business logic and an entity concept, and includes fault location, fault type, and alarm information.

In some embodiments, the expert system module for the dissolved gas in the transformer oil includes a database for dissolved gases in the oil, which includes corresponding fault type labels, and a set of diagnostic algorithms for the dissolved gas in the transformer oil, which are constantly updated in subsequent operation processes.

The transformer temperature distribution diagnosis rule module includes a hot spot temperature empirical formula set and an infrared temperature intelligent diagnosis system.

In some embodiments, the dynamic twin module is specifically configured to execute the following operations.

(a) Different fault locations/fault types/fault severities are set according to diagnostic needs for a transformer model based on software simulation to obtain a dynamic simulation data set.

(b) The dynamic simulation data set is compared to a device history record or real-time monitoring data, a criteria and a threshold are set, and whether an error between the dynamic simulation data set and the device history record or the real-time monitoring data satisfies requirements is determined.

(c) The transformer model is saved when the error requirements are satisfied, and participates in subsequent diagnosis process as a part of the twin, otherwise, returns to Step (a).

(d) During a system operation process, with interaction of the monitoring data and measurement tests, accuracy of the transformer model and the expert system is corrected.

In some embodiments, the decision-making diagnosis module is specifically configured to execute the following operations.

(a) On-site sensory data is preliminarily classified and suitable components of the system support module are searched.

(b) Device information, one or more diagnostic systems and historical data samples corresponding to a sensory data type are extracted.

(c) A dynamic twin module is searched for monitoring information of the simulation model, and device supplementary sample is obtained.

(d) The original diagnosis system in Step (b) is trained using the samples in Steps (b) and (c), and a new diagnosis system for the data to be diagnosed is updated.

(e) The sensory data in Step (a) is inputted into the new diagnosis system of Step (d). A multi-dimensional diagnosis result is outputted and decision-making options are provided when the sensory data is not unique in type.

In some embodiments, the user interface module includes a UI interface, and may provide information query, annotation, modification, and a function to fine-tune the digital twin model.

### BRIEF DESCRIPTION OF THE DRAWINGS

FIG. 1 is a structural block diagram of a transformer fault diagnosis and positioning system based on a digital twin according to an embodiment of the disclosure.

FIG. 2 is a working principle diagram of the transformer fault diagnosis and positioning system based on the digital twin according to an embodiment of the disclosure.

FIG. 3 is a schematic flowchart of a transformer fault diagnosis and positioning method based on a digital twin according to an embodiment of the disclosure.

FIG. 4 is a schematic diagram of a flowchart of dynamic modeling of the transformer in a dynamic twin module according to an embodiment of the disclosure.

FIG. 5 is a schematic diagram of a decision-making diagnosis process according to an embodiment of the disclosure.

### DESCRIPTION OF THE EMBODIMENTS

In order to enhance comprehension of the objectives, technical solutions, and advantages of the disclosure, the disclosure is further described in detail as follows with reference to accompanying drawings and embodiments. It should be understood that the specific embodiments described here are only used to explain the disclosure, and are not meant to limit the disclosure. In addition, the technical features involved in the various embodiments of the disclosure described below may be combined with each other as long as they do not conflict with each other. The disclosure is not only suitable for fault diagnosis and positioning of a transformer, but may also be extended to the field of health management of similar devices.

A transformer fault diagnosis and positioning system based on a digital twin according to the disclosure is configured to fuse transformer historical ledger information, high-dimensional monitoring data, and the dynamic simulation model to generate an interactive digital twin model, which ensures consistency of fault features of virtual and real systems, thereby realizing accurate cognition and closed-loop optimization of fault diagnosis and positioning.

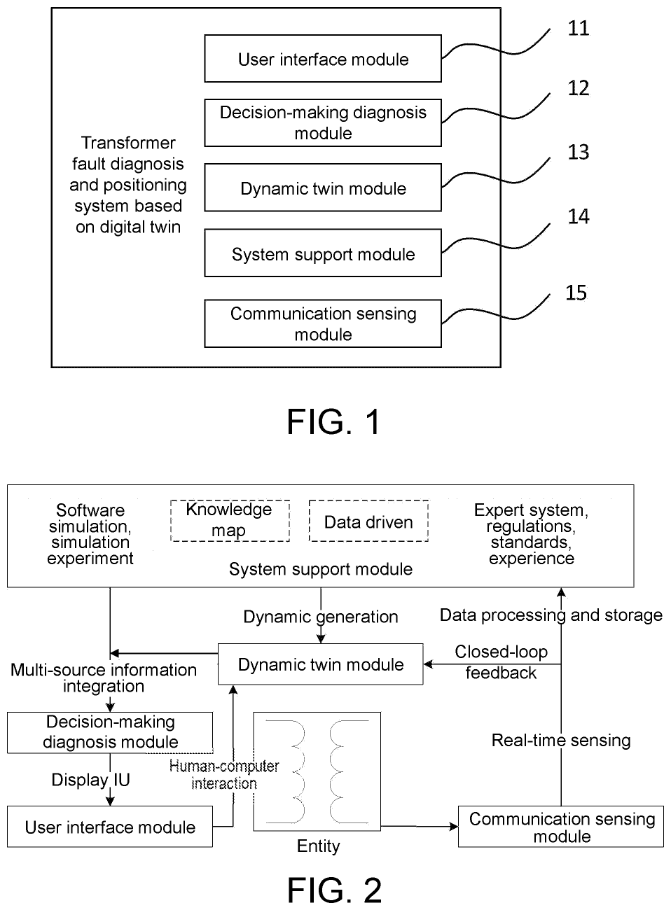

FIG. 1 is a structural block diagram of a transformer fault diagnosis and positioning system based on a digital twin according to an embodiment of the disclosure, which is configured to realize integration, intelligence, and high-accuracy of health management of a transformer device. The system includes the following components.

A communication sensing module 15, which directly obtains bottom-level monitoring data from a device entity, and transmits the obtained bottom-level monitoring data to an upper-level system support module 14 in a wired/wireless manner.

The system support module 14, which receives and preprocesses the bottom-level monitoring data from the communication sensing module 15, and stores various data, models, and expert systems. This module is connected to a dynamic twin module 13 and a decision-making diagnosis module 12, uses a data-driven sample database as a main resource, uses an expert system and a model-driven library as auxiliary resources, and provides basic support to an improved dynamic digital model and reliable decision-making through providing knowledge, expertise, and a data model.

The dynamic twin module 13, which analyzes a multi-dimensional probability status of a device fault, obtains samples containing different device fault locations, fault types, and fault severities through simulation or simulation testing, constructs a degradation model based on different fault types and locations, and realizes dynamic modeling of different health states of the transformer. And then, further realizes model correction through human-computer interaction, real-time measurement, and dynamic update.

In which, the process of constructing the degradation model based on the different fault types and locations may be realized based on conventional simulation or simulation testing, to construct a simulation model with the different fault types and locations as input and a fault state as an output as the degradation model.

The decision-making diagnosis module 12, which with regards to inputted data, integrates information from the system support module 14 and the dynamic twin module 13, constructs a digital twin of data to be diagnosed using the digital twin technology, and realizes diagnosis positioning through fusion of expert expertise, digital simulation, and artificial intelligence.

A user interface module 11, which displays a diagnosis result and related useful information through a user interface, and provides further decision-making options and human-computer interaction.

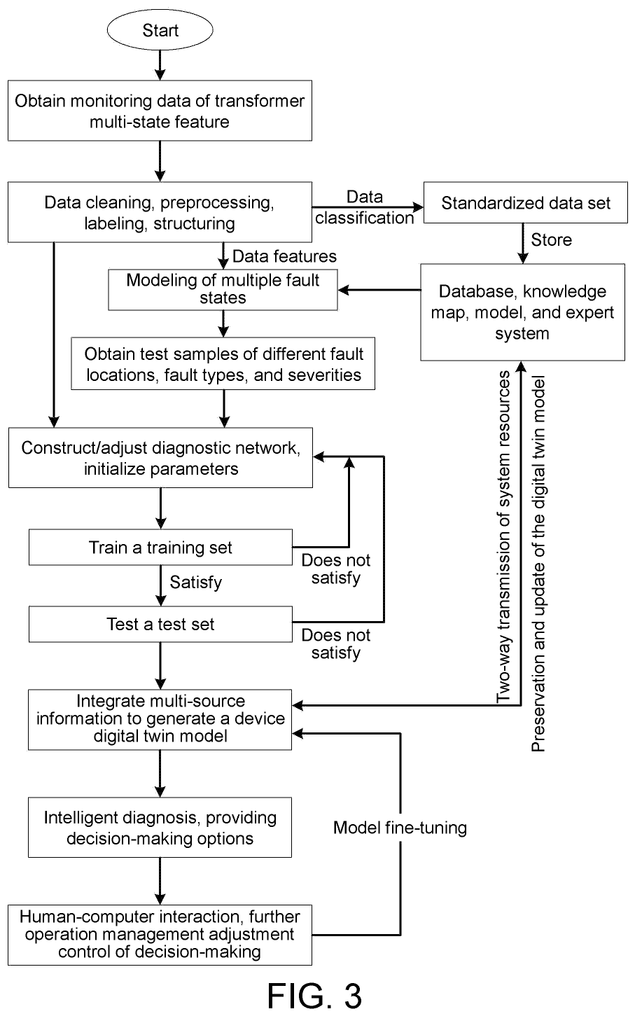

The above-mentioned modules cooperate with each other and have complementary advantages, and their relationships and functions are shown in FIG. 2. The digital twin of the entity is constructed with intelligent sensing as basis, big data storage as support, the expert system as expertise, simulation as auxiliary, standardized criteria as scale, and intelligent decision-making as means, so as to form a diagnostic intelligent system that has closed-loop feedback, and is driven by real-time interactive data. After the system is constructed, a flowchart of a transformer fault diagnosis method based on the digital twin is shown in FIG. 3.

Furthermore, the aforementioned communication sensing module 15 includes a monitoring data acquisition module for a dissolved gas in transformer oil, a transformer temperature monitoring module, a transformer winding deformation monitoring module, a transformer partial electrical discharge monitoring module etc. In which, the transformer temperature monitoring module includes an internal hot spot temperature monitoring and external infrared thermography monitoring. The hot spot temperature monitoring is realized through a sensor, and the infrared thermography is realized through a thermal imager. The transformer winding deformation monitoring module may adopt multiple means such as vibration signal sensing, sweep frequency response analysis, and physical measurement.

Furthermore, the preprocessing function of the above-mentioned system support module 14 is able to use various signal processing methods to analyze original data, which specifically includes data cleaning, feature extraction, labeling, structuring, and so on.

The system support module 14 includes the following components. A transformer ledger knowledge map module, an expert system module for the dissolved gas in the transformer oil, a transformer temperature distribution diagnosis rule module, a transformer winding deformation simulation and diagnosis module, and a partial electrical discharge frequency spectrum characteristic analysis module.

The transformer ledger knowledge map module includes the following 4 parts. (a) A device entity map, that is, basic attributes of the transformer such as rated/operating voltage, power, frequency, capacity, structural parameters, materials, as well as working location, geographic information, environmental temperature and humidity, etc. of the transformer. (b) A device case map, that is, fault history case records handling methods, etc. of a similar transformer, and a similarity between the devices is calculated through structural feature matching. (c) A business logic map, that is, a handling plan or procedure of the transformer, which contains information such as general operating principles, failure causes, and handling points. (d) A concept map, which is intermediate information that connects business logic and an entity concept, such as fault location, fault type, alarm information, etc.

The expert system module for the dissolved gas in the transformer oil includes a database for dissolved gases in the oil (including corresponding fault type labels), and a set of diagnostic algorithms for the dissolved gas in the transformer oil, which are constantly updated in subsequent operation processes. A dissolved gas in the oil algorithm includes commonly encountered an attention value method (Using the IEC typical gas concentration values as thresholds), a ratio method (Rogers Ratio method), a graphic method (Duval Triangle method), an artificial intelligence technology (artificial neural network, deep learning), etc.

The transformer temperature distribution diagnosis rule module includes a hot spot temperature experience formula set and an infrared temperature intelligent diagnosis system, which is able to realize intelligent identification and positioning of faults based on current artificial intelligence.

The transformer winding deformation simulation and diagnosis module includes 2D and 3D simulation models of the transformer, such as a COMSOL model, an Ansys model, as well as a program-based simulation model, and perform diagnosis through a current intelligent algorithm or an empirical indicator.

The partial electrical discharge frequency spectrum characteristic analysis module stores a partial discharge characteristic of the transformer for fault diagnosis.

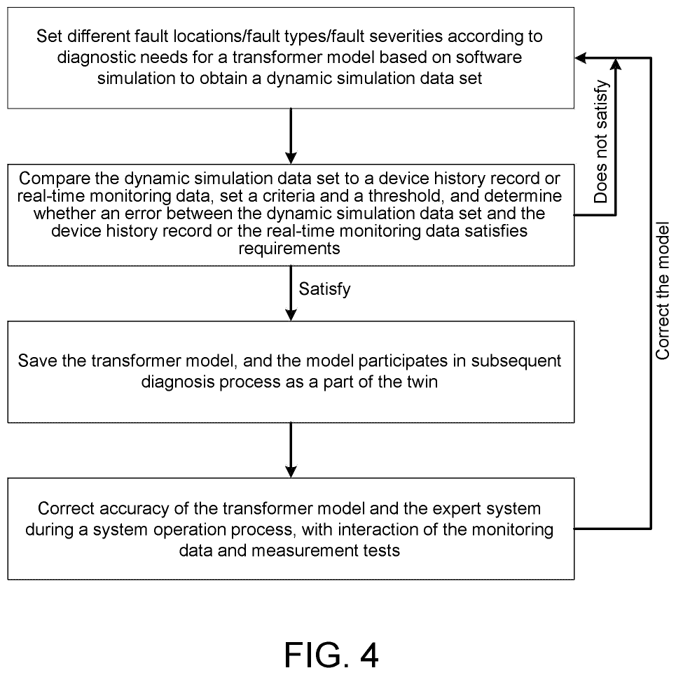

Furthermore, as shown in FIG. 4, the above-mentioned dynamic twin module 13 performs simulation according to the fault severity and probability distribution of the corresponding fault to realize the dynamic modeling of the multiple health states, which includes the following steps.

(a) Different fault locations/fault types/fault severities are set according to diagnostic needs for a transformer model based on software simulation, such as a transformer winding simulation model, to obtain a dynamic simulation data set.

(b) The dynamic simulation data set is compared to a device history record or real-time monitoring data, a criteria and a threshold (for example, an average percentage error is not greater than a certain threshold) are set, and whether an error between the dynamic simulation data set and the device history record or the real-time monitoring data satisfies requirements is calculated.

(c) The transformer model is saved when the error requirements are satisfied, and participates in subsequent diagnosis process as a part of the twin, otherwise, returns to Step (a).

(d) During a system operation process, with the interaction of the monitoring data, measurement tests, etc., accuracy of the simulation model and the expert system is corrected.

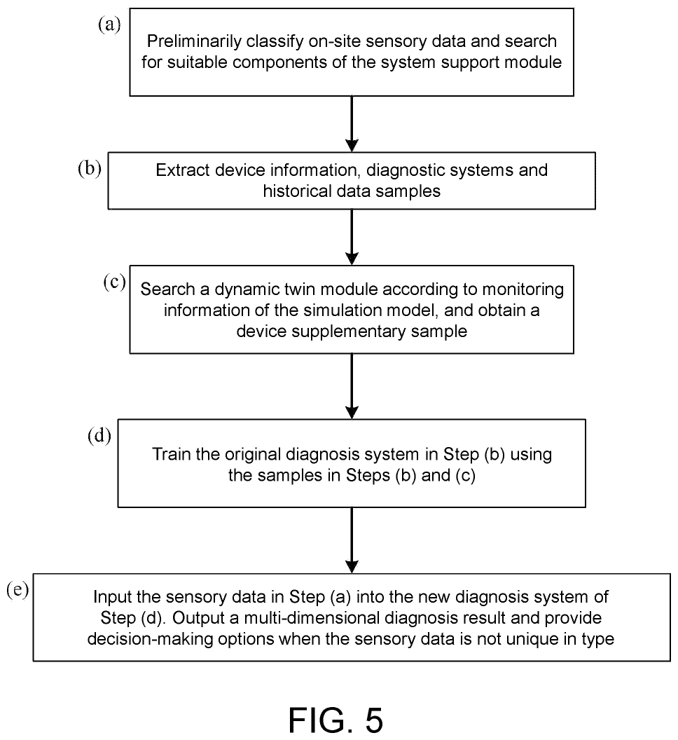

Furthermore, as shown in FIG. 5, the decision-making diagnosis module 12 uses the digital twin technology to construct the digital twin of the data to be diagnosed, which includes the following steps.

(a) On-site sensory data is preliminarily classified and suitable components of the system support module are searched.

(b) Device information, one or more diagnostic systems and historical data samples corresponding to a sensory data type are extracted.

(c) A dynamic twin module is searched for monitoring information of the simulation model, and a device supplementary sample is obtained.

(d) The original diagnosis system in Step (b) is trained using the samples in Steps (b) and (c) through machine learning or deep neural network training, so as to update a new diagnosis system for the data to be diagnosed, that is, the digital twin model in FIG. 3.

(e) The sensory data in Step (a) is inputted into the new diagnosis system of Step (d). A multi-dimensional diagnosis result is outputted and decision-making options are provided when the sensory data is not unique in type.

Furthermore, the user interface module 11 includes a UI interface, and may provide functions such as information query, annotation, modification, and fine-tuning of the digital twin model. The UI interface not only displays the fault diagnosis and location results of the digital twin system, but also provides some basic device information and a visual model, in addition to a user interaction channel, which can facilitate further decision-making and modifications by relevant personnel. The label may be modified for data that a professional thinks that has to be relabeled. In addition, for the rule and model that have to be adjusted, they may be easily adjusted and updated to the database of the system support module.

In general, compared to the related art, the above technical solutions proposed by the disclosure can have the following capabilities.

The disclosure fuses the transformer historical ledger information, high-dimensional monitoring data, and the dynamic simulation model to build a digital twin model capable of real-time situational awareness and super real-time virtual deduction, construct the digital twin of the transformer capable of panoramic monitoring and diagnosis, integrates multiple types of information, makes up for sample shortages, fuses diagnosis results, which helps to realize timely and accurate assessment of the operating status and health state of a device such as the transformer. In addition, intuitive display is possible through the UI interface, decision-making and human-computer interaction are further provided, and an intelligent platform for information storage and sharing is built, which enables closed-loop optimization of the device health management, and improves the economic value and reliability of the transformer device.

It should be noted that according to implementation requirements, each step/component described in the application may be split into more steps/components, or two or more steps/components or partial operations of the steps/components may be combined into new steps/components, so as to realize the purpose of the disclosure.

Although the disclosure has been described with reference to the above-mentioned embodiments, it is not intended to be exhaustive or to limit the disclosure to the precise form or to exemplary embodiments disclosed. It is apparent to one of ordinary skill in the art that modifications to the described embodiments may be made without departing from the spirit and the scope of the disclosure. Accordingly, the scope of the disclosure is defined by the claims appended hereto and their equivalents in which all terms are meant in their broadest reasonable sense unless otherwise indicated.

What is claimed is:

1. A transformer fault diagnosis and positioning system based on a digital twin, comprising:
a communication sensing module, configured to directly obtain bottom-level monitoring data from a device entity, and transmit the obtained bottom-level monitoring data to an upper-level system support module;
the system support module, configured to receive and preprocess the bottom-level monitoring data from the communication sensing module, and store various data, models and expert systems;
a dynamic twin module, configured to analyze a multi-dimensional probability status of a device fault, construct a degradation model based on different fault types and locations, and realize dynamic modeling of different health states of the transformer, and then further realizes model correction through human-computer interaction, real-time measurement, and dynamic update; and
a decision-making diagnosis module, configured to integrate the information from the system support module and the dynamic twin module with data to be diagnosed, and use digital twin technology to construct a digital twin of the data to be diagnosed, and realize diagnosis positioning.

2. The system according to claim 1, wherein the communication sensing module comprises:
a monitoring data acquisition module for a dissolved gas in transformer oil, configured to acquire concentration data of the dissolved gas in the transformer oil;
a transformer temperature monitoring module, configured to monitor an internal hot spot temperature and external infrared thermography;
a transformer winding deformation monitoring module, configured to monitor a transformer winding deformation status; and
a transformer partial electrical discharge monitoring module, configured to monitor a partial electrical discharge status of the transformer.

3. The system according to claim 1, wherein the system support module is configured to analyze the bottom-level monitoring data coming from the communication sensing module using a signal processing method, and comprises data cleaning, feature extraction, labeling, and structuring.

4. The system according to claim 3, wherein the system support module comprises:
a transformer ledger knowledge map module, configured to store transformer historical ledger information;
an expert system module for the dissolved gas in the transformer oil, configured to store an expert system;
a transformer temperature distribution diagnosis rule module, configured to store a temperature distribution diagnosis rule;
a transformer winding deformation simulation and diagnosis module, configured to store a simulation model of the transformer; and
a partial electrical discharge frequency spectrum characteristic analysis module, configured to analyze the fault according to a partial electrical discharge frequency spectrum characteristic.

5. The system according to claim 4, wherein the information stored by the transformer ledger knowledge map module comprises:
a device entity map, comprising rated/operating voltage, power, frequency, capacity, structural parameters, materials, working location, geographical information, and environmental temperature and humidity of the transformer;
a device case map, comprising fault history case records and handling methods of a similar transformer, and calculates a similarity between the two devices through structural feature matching;
a business logic map, comprising a handling plan or a procedure of the transformer, containing general operating principles, fault causes, and handling points; and
a concept map, intermediate information that connects business logic and an entity concept, and comprising fault location, fault type, and alarm information.

6. The system according to claim 4, wherein the expert system module for the dissolved gas in the transformer oil comprises a database for dissolved gases in the oil, comprising corresponding fault type labels, and a set of diagnostic algorithms for the dissolved gas in the transformer oil, which are constantly updated in subsequent operation processes,
wherein the transformer temperature distribution diagnosis rule module comprises a hot spot temperature empirical formula set and an infrared temperature intelligent diagnosis system.

7. The system according to claim 1, wherein the dynamic twin module is specifically configured to:
(a) set different fault locations/fault types/fault severities according to diagnostic needs for a transformer model based on software simulation to obtain a dynamic simulation data set;
(b) compare the dynamic simulation data set to a device history record or real-time monitoring data, set a criteria and a threshold, and determine whether an error between the dynamic simulation data set and the device history record or the real-time monitoring data satisfies requirements;
(c) save the transformer model when the error requirements are satisfied to participate in subsequent diagnosis process as a part of the twin, or otherwise, return to Step (a); and
(d) correct accuracy of the transformer model and the expert system during a system operation process, with interaction of the monitoring data and measurement tests.

8. The system according to claim 1, wherein the decision-making diagnosis module is specifically configured to:
(a) preliminarily classify on-site sensory data and search for suitable components of the system support module;
(b) extract device information, one or more diagnostic systems and historical data samples corresponding to a sensory data type;
(c) search a dynamic twin module for monitoring information of the simulation model and obtain a device supplementary sample;
(d) train the original diagnosis system in Step (b) using the samples in Steps (b) and (c), and update a new diagnosis system for the data to be diagnosed; and
(e) input the sensory data in Step (a) into the new diagnosis system of Step (d), and output a multi-dimensional diagnosis result and provide decision-making options when the sensory data is not unique in type.

9. The system according to claim 1, further comprising:
a user interface module, configured to display a diagnosis result and related information through a user interface, and provide further decision-making options and human-computer interaction.

10. The system according to claim 9, wherein the user interface module comprises a user interface, and is able to provide information query, annotation, modification, and a function to fine-tune the digital twin model.
